(19) United States
(12) Patent Application Publication
(10) Pub. No.: US 2016/0008038 A1
(43) Pub. Date: Jan. 14, 2016

(54) METHODS AND SYSTEMS FOR CONSTRAINT OF MULTIPLE SPINE SEGMENTS

(71) Applicant: Simpirica Spine, Inc., San Carlos, CA (US)

(72) Inventors: Todd Alamin, Woodside, CA (US); Ian Bennett, San Francisco, CA (US); Louis Fielding, San Carlos, CA (US); Colin Cahill, Portola Valley, CA (US); Jeffrey Schwardt, Palo Alto, CA (US)

(21) Appl. No.: 14/673,546

(22) Filed: Mar. 30, 2015

Related U.S. Application Data
(63) Continuation of application No. 13/274,171, filed on Oct. 14, 2011, now abandoned.
Application No. 13/274,171 is a continuation of application No. PCT/US2010/031471, filed on Apr. 16, 2010.

(51) Int. Cl. A61B 17/70

(52) U.S. Cl. CPC A61B 17/7062; A61B 17/7022; A61B 2017/564

(57) ABSTRACT

Methods, apparatus and systems for constraining spinous processes to elastically limit flexion of two or more adjacent spinal segments rely on placing a tether structure over at least three adjacent vertebral bodies or two adjacent vertebral bodies and the sacrum. The tether structures may be continuous, for example in the form of a continuous loop, or may be discontinuous, for example in the form of a loop or elongate element having at least two anchor structures for securing in bone.

The present application is a continuation of U.S. patent application Ser. No. 13/274,171 (Attorney Docket No. 41564-703.301) filed Oct. 14, 2011 which is a continuation of International PCT Application No. PCT/US2010/031471 (Attorney Docket No. 41564-703.606) filed Apr. 16, 2010, which claims priority to U.S. patent application Ser. No. 12/426,119 (Attorney Docket No. 41564-703.503) filed Apr. 17, 2009, which is a continuation in part of International PCT Application No. PCT/US2007/081822 (Attorney Docket No. 41564-703.605) filed Oct. 18, 2007, which claims priority to U.S. Provisional Patent Application No. 60/862,085 (Attorney Docket No. 41564-703.101) filed Oct. 19, 2006; U.S. patent application Ser. No. 12/426,119 (Attorney Docket No. 41564-703.503) is also a non-provisional of, and claims the benefit of U.S. Provisional Patent Application No. 61/158,892 (Attorney Docket No. 41564-713.101) filed Mar. 10, 2009; the entire contents of each of which are incorporated herein by reference.

### BACKGROUND OF THE INVENTION

1. Field of the Invention

The present invention relates generally to medical methods and apparatus. More particularly, the present invention relates to methods and devices for restricting spinal flexion in patients having back pain or other spinal conditions.

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine (FIG. 1). Pain tends to be exacerbated when patients put their lumbar spines into flexion (i.e. by sitting or bending forward) and relieved when they put their lumbar spines into extension (i.e. arching backwards). Discogenic pain can be quite disabling, and for some patients, can dramatically affect their ability to work and otherwise enjoy their lives.

This pain experienced by patients with discogenic low back pain can be thought of as flexion instability, and is related to flexion instability that is manifested in other conditions. The most prevalent of these is spondylolisthesis, a spinal condition in which abnormal segmental translation is exacerbated by segmental flexion. The device described here should as such also be useful for these other spinal disorders associated with segmental flexion, for which the prevention or control of spinal segmental flexion is desired.

Current treatment alternatives for patients diagnosed with chronic discogenic pain are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion is not usually recommended for discogenic pain because it is irreversible, costly, associated with high morbidity, and of questionable effectiveness. Despite its drawbacks, however, spinal fusion for discogenic pain remains common due to the lack of viable alternatives.

Recently, a less invasive and potentially more effective treatment for discogenic pain has been proposed. A spinal implant has been designed which inhibits spinal flexion while allowing substantially unrestricted spinal extension. The implant is placed over one or more adjacent pairs of spinal processes and provides an elastic restraint to the spreading apart of the spinal processes which occurs during flexion. Such devices and methods for their use are described in U.S. Patent Publication No. 2005/0216017A1, published on Sep. 29, 2005, (now U.S. Pat. No. 7,458,981) and having common inventors with the present application. The entire contents of U.S. Patent Publication No. 2005/0216017 A1 are incorporated herein by reference.

As illustrated in FIG. 2, an implant 10 as described in the '017 publication, typically comprises an upper strap component 12 and a lower strap component 14 joined by a pair of compliance members 16. The upper strap 12 is shown disposed over the top of the spinous process SP4 of L4 while the lower strap 14 is shown extending over the bottom of the spinous process SP5 of L5. The compliance member 16 will typically include an internal element, such as a spring or rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinal processes which provides a force that resists flexion without substantially limiting extension of the segment. The force increases as the processes move further apart. Usually, the straps themselves will be essentially non-compliant so that the degree of elasticity or compliance may be controlled and provided solely by the compliance members 16.

Although providing significant benefits, the system illustrated in FIG. 2 is intended to treat only a single spinal segment between a pair of adjacent vertebral bodies. In some patients, it would be desirable to treat two or more successive spinal segments.

For these reasons, it would be desirable to provide improved spinal implants, implant systems, and methods for their use for limiting flexion in two or more successive spinal segments. It would be particularly desirable if the implants, systems, and methods permitted the spinous processes of three or more adjacent vertebral bodies, or two adjacent vertebral bodies and the sacrum, to be elastically coupled using a single implant structure which can constrain multiple adjacent spinal features. At least some of these objectives will be met by the inventions described hereinbelow.

2. Description of the Background Art

US 2005/0216017A1 has been described above. Other patents and published applications of interest include: U.S. Pat. Nos. 4,966,600; 5,011,494; 5,092,866; 5,116,340; 5,282,863; 5,395,374; 5,415,658; 5,415,661; 5,449,361; 5,456,722; 5,462,542; 5,496,318; 5,540,698; 5,609,634; 5,645,599; 5,725,582; 5,902,305; Re. 36,221; 5,928,232; 5,935,133; 5,964,769; 5,989,256; 6,053,921; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; Published U.S. Patent Application Nos. US 2002/0151978; US 2004/0024458; US 2004/0106995; US 2004/0116927; US 2004/0117017; US 2004/0127989; US 2004/0172132; US 2005/0033435; US 2005/0049708; US 2006/0069447; Published PCT Application Nos. WO 01/28442 A1; WO 02/03882 A2; WO 02/051326 A1; WO 02/071960 A1; WO 03/045262 A1; WO 2004/052246 A1; WO 2004/073532 A1; and Published Foreign Application Nos. EP 0322334 A1; and FR 2 681 525 A1.

### BRIEF SUMMARY OF THE INVENTION

The present invention provides spinal implants, implant systems, and methods for constraining spinous processes to elastically limit flexion of two or more adjacent spinal segments. As used herein, the phrase “spinal segment” is synonymous with the phrase “functional spinal unit (FSU)” and intended to mean the smallest physiological motion unit of the spine that exhibits biomechanical characteristics similar to those of the entire spine. A spinal segment or FSU consists of two adjacent vertebrae, the intervertebral disc and all adjoining ligaments between them and excludes other connecting tissues such as muscles. The three-joint complex that results is sometimes referred to as the “articular triad.” Another term for the FSU is spinal motion segment. These definitions are taken from White A A, Panjabi M M. (1990), Clinical Biomechanics of the Spine, Philadelphia, J B Lippincott. The methods comprise placing a tether structure over the spinous processes of at least three adjacent vertebral bodies, or over the spinous processes of two adjacent vertebral bodies and a sacrum, wherein the structure elastically couples the at least two non-adjacent spinous processes or one spinous process and a non-adjacent sacrum. The spinous processes and optionally a sacrum can be interconnected and elastically coupled in a variety of ways.

In a first exemplary pattern, the tether structure elastically couples an upper spinous process to a lower spinous process, or to the sacrum, with at least one intermediate spinous process being free from coupling. In an alternative pattern, the tether structure elastically couples an upper spinous process and a lower spinous process or sacrum, as well as the at least one intermediate spinous process. The spinous processes and optionally the sacrum may be elastically coupled by a single contiguous tether structure, or in other embodiments may be elastically connected by two or more contiguous tether structures. In the case of two or more contiguous tether structures, the tether structures may further be interconnected, coupled, or linked in order to provide desired elastic restraint characteristics. The spinous processes being connected will typically be in the lumbar region, most typically being at the lower levels of the lumbar, and even more particularly being at L3, L4, L5 and the sacrum. In most instances, the spinous processes, and optionally the sacrum, are elastically coupled to inhibit flexion with the spaces between the adjacent vertebral bodies being free from structure which would substantially limit or inhibit extension of the spinal segments being treated. A first portion and a second portion of the tether structure may extend between the upper spinous process and the lower spinous process or the sacrum. The first and second portions of the tether structure may be disposed symmetrically on opposite sides of the spinous processes and they also may be parallel to one another.

In another aspect of the present invention, a spinal implant comprises a contiguous tether structure adapted to circumscribe at least two non-adjacent spinous processes, or in other instances, to an anchor location on the sacrum and one non-adjacent spinous process. At least a portion of the tether structure will provide an elastic resistance to elongation in response to an elongation force which results from flexion of the spinal segments between the non-adjacent spinous processes and/or between the one non-adjacent spinous process and the sacrum. The tether structure limits flexion therebetween without substantially limiting extension therebetween. A first portion and a second portion of the tether structure may extend between the non-adjacent spinous processes or between the one non-adjacent spinous process and the sacrum. The first and second portions of the tether structure may be disposed symmetrically on opposite sides of the spinous processes and they also may be parallel to one another. Often, the implant will include at least two compliance members positioned as part of the tether structure such that they will lie symmetrically on opposite sides of the spinous processes when implanted. In still other embodiments, the contiguous tether structures will include at least four such compliance members. The compliance members will typically be coupled to non-compliant and/or cable components of the tether structure so that it is the compliance members which provide most or all of the compliance or elasticity in the implants. Exemplary compliance structures are illustrated in copending U.S. Patent Publication No. 2005/02161017 A1 (now U.S. Pat. No. 7,458,981).

In some embodiments, the contiguous tether structure will be continuous so that the structure forms a loop which may be placed over the non-adjacent spinous processes. Such continuous “loop” tether structures will usually be maintained on the spinous processes by friction and interference fit, but in some cases could be modified to permit further attachment by stapling, welding, gluing, suturing, or the like. In other embodiments, the contiguous tether structure will be discontinuous and will have two ends which are adapted for anchoring for direct attachment to the bone. Such discontinuous tether structures will be suitable for anchoring in the sacrum.

In a third aspect of the present invention, systems comprising a spinal implant as generally described above further include at least one additional contiguous tether structure. The additional tether structure will usually be adapted to circumscribe two adjacent or non-adjacent spinous processes or a sacrum. The additional contiguous tether structures may be continuous so that they can be looped over the spinous processes, or in other instances may be discontinuous and have two ends adapted for anchoring directly in the bone. The additional contiguous tether structure may be interconnected with the primary tether structure but will frequently be formed separately.

In still another aspect of the present invention, a method for constraining spinous processes to elastically limit flexion of two or more adjacent spinal segments comprises placing a first tether structure over a superior spinous process and an inferior spinous process of a first spinal segment. The first tether structure elastically couples the superior spinous process and the inferior spinous process so as to limit flexion therebetween without substantially limiting extension thereof. A first portion of the first tether structure extends between the superior spinous process and the inferior spinous process of the first spinal segment, and a second portion of the first tether structure extends between the superior spinous process and the inferior spinous process of the first spinal segment. The first and the second portions are disposed symmetrically on opposite sides of the spinous processes, and they are substantially parallel to one another. The method also comprises placing a second tether structure over a superior spinous process and an inferior spinous process or a sacrum of a second spinal segment. The second tether structure elastically couples the superior spinous process and the inferior spinous process or the sacrum of the second spinal segment so as to limit flexion therebetween without substantially limiting extension thereof. A first portion of the second tether structure extends between the superior spinous process and the inferior spinous process or the sacrum of the second spinal segment, and a second portion of the second tether structure extends between the superior spinous process and the inferior spinous process or the sacrum of the second spinal segment. The first and the second portions are disposed symmetrically on opposite sides of the spinous processes, and they are substantially parallel to one another. The first spinal segment is adjacent and superior to the second spinal segment, and one of the first or second tether structures is positioned anteriorly relative to the other tether structure.

One of the first or the second tether structures may be positioned anteriorly relative to the other tether structure. The first spinal segment and the second spinal segment may share a common spinous process such as when the inferior spinous process of the first spinal segment and the superior spinous process of the second spinal segment are the same spinous process. Placing the first tether structure and placing the second tether structure may comprise placing the tether structures without engaging or overlapping one another. Placing the first tether structure and placing the second tether structure may comprise looping the tether structures through each other whereby the tether structures encircle the common spinous process. The first tether structure and the second tether structures may each comprise a discrete contiguous loop. At least one of the tether structures may comprise two compliance members, and the compliance members may be positioned symmetrically to lie on opposite sides of the spinous processes.

In another aspect of the present invention, a system for restricting flexion of two or more adjacent spinal segments comprises a first contiguous tether structure and a second contiguous tether structure. The first tether structure comprises an upper tether portion and a lower tether portion. The first tether structure is configured to surround a first spinal segment having a superior spinous process and an inferior spinous process. The upper tether portion is positioned around the superior spinous process, and the lower tether portion is positioned around the inferior spinous process. The first tether structure provides an elastic resistance to flexion of the first spinal segment without substantially limiting extension thereof. The second contiguous tether structure comprises an upper tether portion and a lower tether portion. The second tether structure is configured to surround a second spinal segment having a superior spinous process and an inferior spinous process or a sacrum. The upper tether portion of the second tether structure is positioned around the superior spinous process of the second spinal segment, and the lower tether portion of the second tether structure is positioned around the inferior spinous process of the second spinal segment or coupled to the sacrum. The second tether structure provides an elastic resistance to flexion of the second spinal segment without substantially limiting extension thereof.

The first tether structure may comprise a first side portion, and a second side portion. The first side portion and the second side portions may extend from the upper spinous process and the lower spinous process of the first spinal segment. The first and the second side portions may be disposed symmetrically on opposite sides of the spinous processes, and may be substantially parallel to one another. The second tether structure may also comprise a first side portion, and a second side portion, which generally take the same form as those in the first tether structure. The first tether structure may be discrete and unconnected to the second tether structure. The first tether structure may be disposed anterior to the second tether structure or posterior thereto. A portion of the first tether structure may overlap with a portion of the second tether structure. The first tether structure may overlap with the second tether structure such that the tether structures loop through each other, and each of the tether structures may be engaged with a common spinous process. The tether structures may overlap and engage the common spinous process at substantially the same distance posterior from an origin of the common spinous process relative to the lamina. The first or the second tether structure may comprise a first and a second compliance member. The compliance members may be disposed symmetrically on opposite sides of the spinous processes, and substantially parallel to one another. The first compliance member may comprise a first elasticity, and the second compliance member may comprise a second elasticity. The first elasticity may be the same as, or different from or the second elasticity. The elasticity of the first and the second compliance members of the first tether structure may be different than the elasticity of the first and the second compliance members of the second tether structure. The first spinal segment and the second spinal segment may share a common spinous process such as when the inferior spinous process of the first spinal segment is the same as the superior spinous process of the second spinal segment.

In some embodiments, the tether structure may be disposed around a first surface of the a spinous process and a second tether structure may be positioned around a second surface of the spinous process, opposite the first surface. The two tethers may be positioned on the spinous process such that one tether is anteriorly disposed on the spinous process relative to the other tether structure.

In other embodiments, the tether structure may have a first compliance member with a first elasticity and a second compliance member with a second elasticity different than the first elasticity. The tether structure may also comprise a first pair of compliance members and a second pair of compliance members. Each of the first pair may have a first elasticity and each of the second pair may have a second elasticity. The first elasticity may be the same or different than the second elasticity. The first pair of compliance members may be superior to the second pair of compliance members.

In preferred embodiments, the tether structure inhibits or limits flexion of a spinal segment without substantially limiting extension therebetween. Thus, in some embodiments, the tether structure may have an elastic stiffness in compression below 3 N/mm and in other embodiments the elastic stiffness in compression may be below 0.5 N/mm.

In still other embodiments, the tether structure may be positioned over an upper spinous process, a lower spinous process and an intermediate spinous process disposed therebetween. The tether structure may comprise a first loop encircling the lower spinous process and the intermediate spinous process so as to substantially prevent flexion therebetween, and the tether structure may also comprise a second loop superior to the first loop. The second loop may have one or more compliance members and may be disposed over the upper spinous process and coupled with the first loop so as to provide a force resistant to flexion of a superior spinal segment relative to the inferior spinal segment.

These and other embodiments are described in further detail in the following description related to the appended drawing figures.

### BRIEF DESCRIPTION OF THE DRAWINGS

FIG. 1 is a schematic diagram illustrating the lumbar region of the spine including the spinous processes (SP), facet joints (FJ), lamina (L), transverse processes (TP), and sacrum (S).

FIG. 2 illustrates a spinal implant of the type described in US 2005/0216017A1.

FIG. 3 illustrates a contiguous tether structure constructed in accordance with the principles of the present invention and adapted for placement over three adjacent spinous processes.

FIG. 4 illustrates a contiguous tether structure similar to that shown in FIG. 3 which further includes four symmetrically placed compliance structures.

FIG. 5 illustrates a contiguous tether structure constructed in accordance with the principles of the present invention which is adapted for placement over three adjacent spinous processes and which further includes an intermediate loop segment for engaging the intermediate spinous process.

FIG. 6 illustrates a contiguous tether structure similar to that shown in FIG. 5, where the intermediate loop structure is adjustably attached to the main tether structure.

FIG. 7 illustrates a contiguous tether structure constructed in accordance with the principles of the present invention and adapted for placement over four adjacent spinous processes including two intermediate loop structures and six symmetrically placed compliance members.

FIG. 8 illustrates yet another contiguous tether structure constructed in accordance with the principles of the present invention comprising two loop segments joined together by connectors adjacent an intermediate spinous process.

FIG. 8A illustrates another embodiment of a tether structure constructed in accordance with the principles of the present invention comprising two loop segments.

FIG. 9 illustrates a contiguous tether structure constructed in accordance with the principles of the present invention and having a discontinuous structure with two ends adapted for anchoring in the sacrum.

FIG. 10 illustrates a system constructed in accordance with the principles of the present invention and including two contiguous tether structures which may be used simultaneously.

FIGS. 11A-11H illustrate various configurations for two constraint structures used to restrict flexion of two spinal segments.

FIGS. 12A-12C illustrate still other configurations for two or more constraint structures used to restrict flexion of two or more spinal segments.

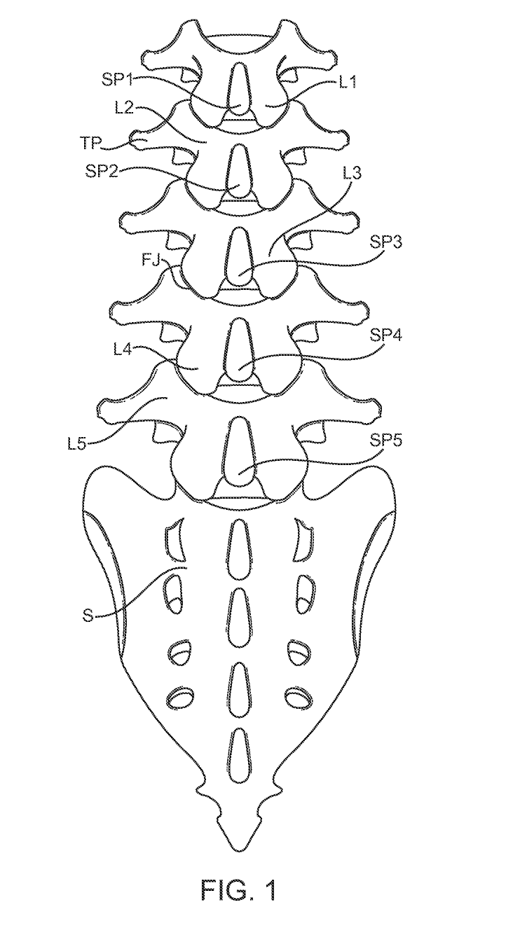

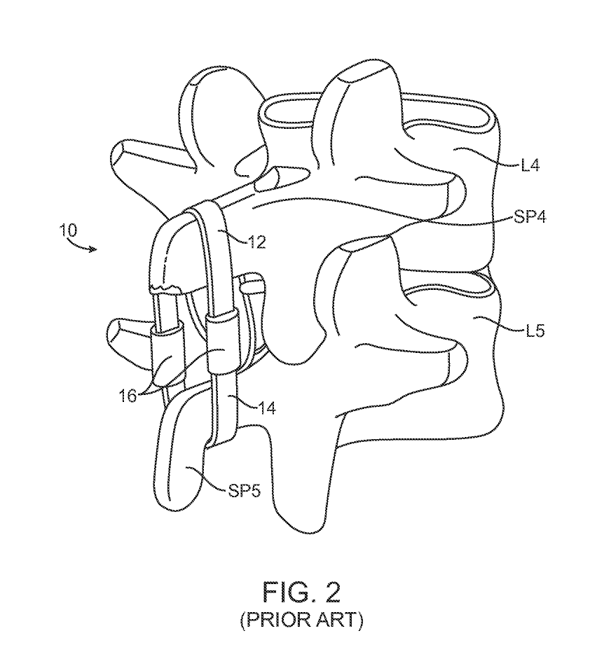

### DETAILED DESCRIPTION OF THE INVENTION

The present invention provides methods, devices, and systems for constraining the flexion of two or more adjacent spinal segments by elastically restraining two or more spinous processes or at least one spinous process and an anchor region on a sacrum. Such restraint is achieved using a tether structure which spans at least three spinous processes or a pair of spinous processes and the sacrum (more specifically, the spinous processes on L4 and L5 as well as an anchor region on the sacrum). The tethers used will typically be in the form of a contiguous tether structure. By “contiguous” it means that the tether may comprise one or more elongate component(s), such as strap(s), cable(s), ribbon(s), or the like, which may be constructed or modified to provide for a desired elastic coupling of one or more spinous processes and optionally an anchor location on the sacrum. Alternatively, the “contiguous” tether structures may comprise a plurality of components, such as the straps, bands, cables, or the like, as mentioned above, together with compliance structures which provide for the desired elastic coupling. In the latter case, the straps, etc., will typically be non-compliant, effecting little or no elongation in response to tension, while the compliance members will provide the desired level of elastically coupling. Combinations of compliant elongate components and separate compliance members will also be possible.

The contiguous tether structures may be continuous or discontinuous. The “continuous” contiguous tether structures will typically be formed into a loop so that the loop may be placed over a pair of spinous processes, typically non-adjacent spinous processes separated by at least one intermediate spinous process. The “discontinuous” contiguous tether structures, in contrast, will have at least two free ends adapted with anchor structures for anchoring to bone, typically to anchor regions on a sacrum.

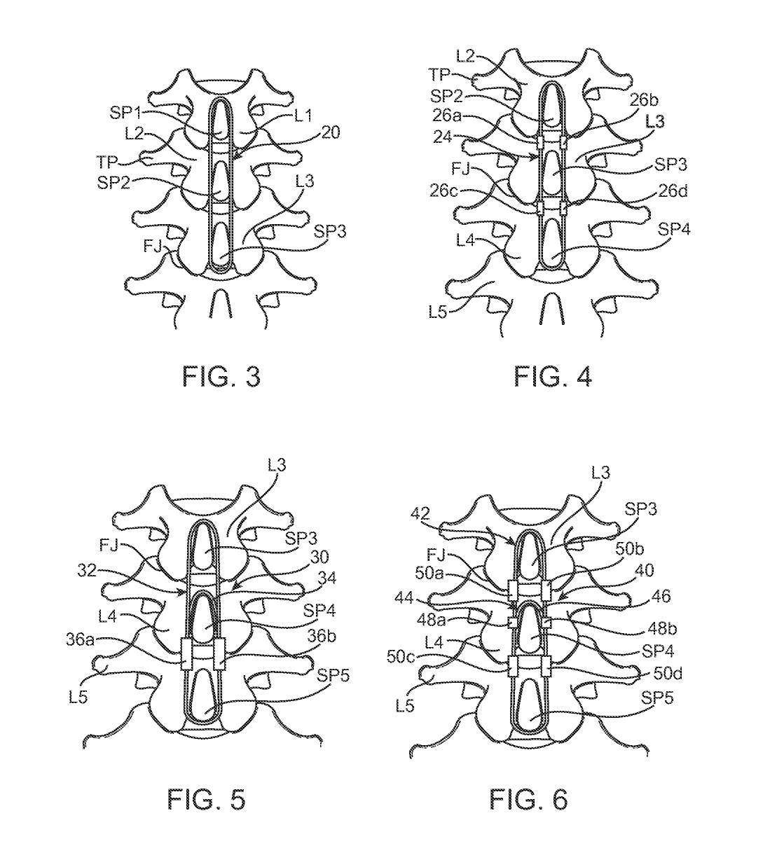

Referring now to FIG. 3, a first exemplary continuous tether structure 20 is shown circumscribing the spinous processes SP1-SP3 on the L1-L3 vertebral bodies. The tether structure 20 may be a simple band, strap, or cable which is formed into a continuous loop, where at least a portion of the structure provides a desired elasticity to inhibit flexion of the spinal segments between L1 and L2 and L2 and L3 in a controlled manner. Elasticity may be provided through use of an elastomeric material, inclusion of spring-like or elastic regions in an otherwise inelastic or non-compliant structure, or the like.

Referring now to FIG. 4, a second exemplary continuous tether structure 24 is similar to tether 20, except that it is provided with separate compliance structures 26a-26d arranged symmetrically on opposite sides of the “ridge” of spinous processes. The tether structure 24 is shown placed on the spinous processes SP2-SP4 on vertebral bodies L2-L4, it will be appreciated that tether structures 20 and 24 could be placed on any three contiguous spinous processes, typically in the lumbar region. Various combinations of elasticities may be used amongst the four compliance members 26a-26d. For example, all four compliance members may have the same elasticity. Alternatively, all four compliance members may have an elasticity different from one another. In some embodiments, the two superior compliance members 26a, 26b may have a first elasticity and the two inferior compliance members 26c, 26d may have a second elasticity different than the first. This allows the resistance to flexion to be varied at different levels of the spinal segment. In still other embodiments, at one motion segment level, the elasticity of a left compliance member 26a may be different than the right compliance member 26b. One of skill in the art will appreciate that any combination of elasticities may be employed in a tether structure having multiple compliance members. This applies to any of the embodiments disclosed herein having multiple compliance members.

The continuous tether structures of the present invention may be formed in multiple interconnected loops, as shown, for example, in FIGS. 5-8. The multiple loops will usually include an outer or peripheral loop which encircles or otherwise engages at least three or more adjacent spinous processes. One or more inner loops may also be provided to engage or encircle one, two, or possibly more of “intermediate” spinous processes within the group which is being restrained.

For example, in FIG. 5, a continuous tether structure 30 includes an outer loop 32 which encircles three adjacent spinous processes, shown as SP3-SP5 on vertebral bodies L3-L5. An inner loop 34 is provided which encircles only SP4 and SP5. The upper portions of the two loops 32 and 34 are both connected in compliance members 36a and 36b. The compliance members may be configured to apply a generally equal elastic tensioning to the upper loop portions as the spinal segments undergo flexion. Alternatively, the compliance members 36a and 36b could be configured to provide different elastic tensioning forces to the upper segments of loops 32 and 34, respectively.

Continuous tether structure 40, as shown in FIG. 6, also comprises an outer loop 42 (shown to encircle SP3-SP5) and an inner loop 44 (shown to encircle SP4 and SP5 only), similar to the tether structure 30 of FIG. 5. An upper loop portion 46, however, is shown attached to sliding attachment members 48a and 48b, which attachment members allow the upper loop structure 46 to be tightened or “cinched” over the top of SP4. The tether structure 40 is also shown with four symmetrically placed compliance members 50a-50d, but it will be appreciated that the tether structure could include only two or even no compliance members, while retaining the adjustably placed upper loop structure 46. As discussed above, any combination of elasticities may be used amongst the compliance members.

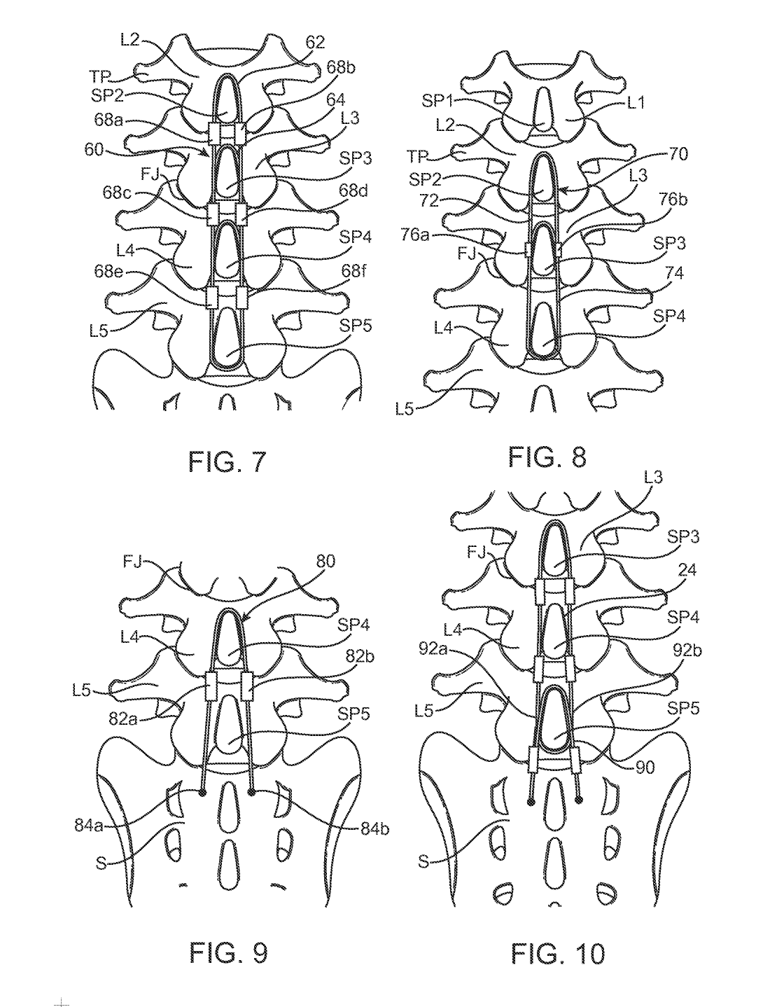

A more complex continuous tether structure 60 including one external loop and two internal loops is illustrated in FIG. 7. The external loop is configured to circumscribe four adjacent spinous processes (SP2-SP4) while the first internal loop defined by loop segment 64 extends over SP3 and a second internal loop segment 66 extends over SP4. Six compliance members 68a-68f are provided symmetrically on opposite sides of the spinous processes, and the ends of the first upper loop segment 64 are connected to compliance members 68c and 68d, respectively, while the ends of the second loop segment 66 are connected to compliance members 68e and 68f, respectively. It will be appreciated that the use of six compliance members and the two intermediate loop segments allows the tension on each of the spinous processes to be independently adjusted to some extent. The overall compliance and elastic force applied to the spinal segments, however, will depend on the cumulative value of the elastic forces provided by all of the compliance members. Thus, elasticity may be varied amongst the compliance members, as previously discussed above.

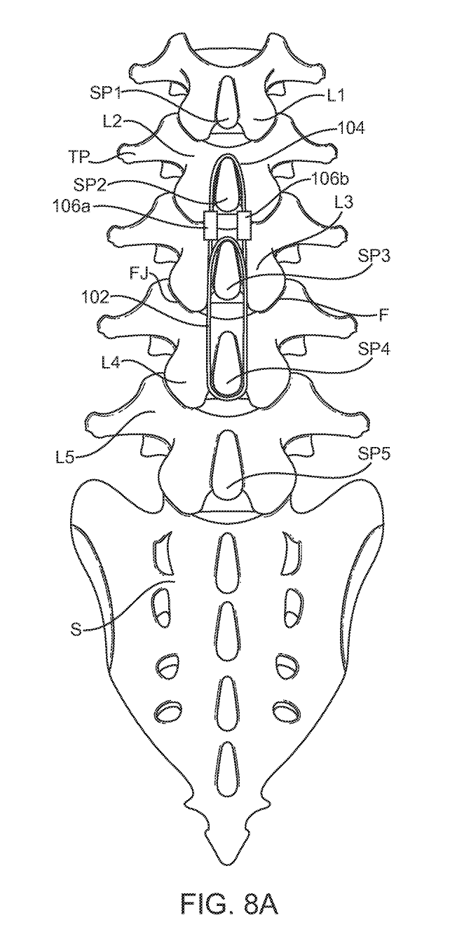

A multiple loop tether structure 70 having a more simple configuration is shown in FIG. 8. An upper loop 72 is adapted to circumscribe a pair of adjacent spinous processes (shown as SP2 and SP3) while a lower loop is adapted to circumscribe an overlapping pair of spinous processes (shown as SP3 and SP4). The two loops are joined by connector components 76a and 76b which may be simple clips or crimps to hold the loops 64 and 66 together (in which case the loops would likely be elastic or partially elastic to allow for controlled flexion of the spinal segments) or could be compliance members which provide for controlled, elastic movement of the upper loop 72 relative to the lower loop 74. In the latter case, the loops would likely be non-compliant.

FIG. 8A illustrates another embodiment similar to that of FIG. 8, except here the tether structure is coupled to two adjacent spinous processes at a first level of the spinal segment and another portion of the tether structure having compliance members is then coupled to a superior spinous process so that flexion is restricted in the suprajacent segment. This may be used, for example, when the spinal segment is fused. In FIG. 8A, a first part of the tether structure consists of a tether 102 circumscribing two adjacent spinous processes SP3-SP4. The tether 102 is disposed around a superior surface of a superior spinous process SP3 and also around an inferior surface of an inferior spinous process SP4. A fusion according to methods known in the art has been performed to fuse L3-L4 together at, or across the level designated by F and therefore tether 102 will often be substantially inelastic in order to prevent flexion between L3-L4 thereby facilitating the fusion F, although some micromotion is still permitted. The tether structure also has a second tether 104 disposed around a superior surface of a superior spinous process SP2 superior to the fused region. The ends of the second tether 104 are coupled with the first part of the tether structure 102, or in alternative embodiments, the ends of the second tether 104 are continuous forming a closed loop and thus are disposed under the inferior surface of SP3. Compliance members 106a, 106b provide a force resistant to flexion of the L2-L3 motion segment supradjacent to the fused region F. This helps to more evenly distribute and possibly lessen loading applied to the fused region, to the level superior to the fused region, and to tethers 102 and 104. The tether structure may also help to reduce excessive motion. Additional details on the use of a tether structure concomitantly with fusion are disclosed in U.S. patent application Ser. No. 12/721,198 (Attorney Docket No. 026398-001310US) and Ser. No. 12/721,238 (Attorney Docket No. 026398-001410US), both filed on Mar. 10, 2010, and both of which the entire contents are incorporated herein by reference.

The contiguous tether structures of the present invention will not always have a continuous structure. As shown in FIGS. 9 and 10, the tether structures may also have a discontinuous geometry including at least two ends adapted to anchor to bone, typically to a surface of the sacrum which generally lacks structure for attaching the lower end of a loop. As shown in FIG. 9, an exemplary discontinuous tether structure 80 comprises a U-shaped tether or band structure including compliance members 82a and 82b. A pair of anchor structures 84a and 84b are provided on two ends of the tether structure 80 and are adapted to be anchored into the face of the sacrum S, as illustrated. In this way, the tether structure 80 can provide for controlled elastic restraint of the spinal segments between SP4 and SP5 and between SP5 and the sacrum. Additional details on sacral attachment may be found in International PCT Application No. PCT/US2010/022767 (Attorney Docket No. 026398-001210PC), filed Feb. 1, 2010, and U.S. patent application Ser. No. 11/827,980 (Attorney Docket No. 026398-000120US), filed Jul. 13, 2007. The entire contents of each of these applications is incorporated herein by reference.

FIG. 10 illustrates a system including a tether structure 24, generally as described with reference to FIG. 4 above, and a second tether structure 90 which is similar to tether structure 80, except that it is adapted only to extend around a single spinous process (SP5) and to be anchored into the sacrum S. Attachment may be provided in a variety of ways as described in copending patent application Ser. No. 11/827,980 and PCT/US2010/022767, both previously incorporated herein by reference. The second tether structure may be attached using a dowel implanted in the sacrum, using alar screws, using superior articular facet screws, using toggle anchors (T-tags) placed in holes formed in a superior articular facet of S1, using hooks attached to the dorsal S1 foramen, or the like. The tether structure 24 and tether structure 90 could be deployed without any interconnection, as generally shown in FIG. 10. Often, however, it might be desirable to interconnect the tether structures at their crossover points 92a, 92b, generally adjacent to the two sides of SP5. The attachment could be accomplished using a crimp structure (not shown) or by otherwise tying, welding, or fusing the tether structures together.

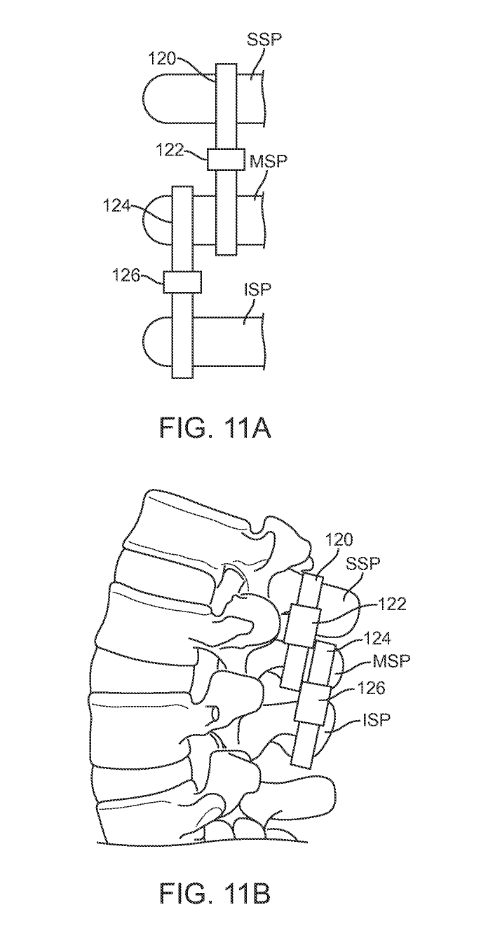

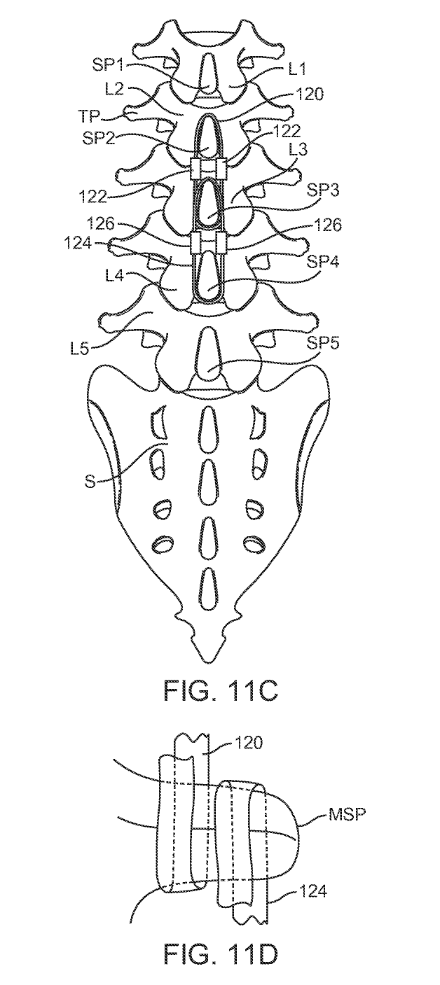

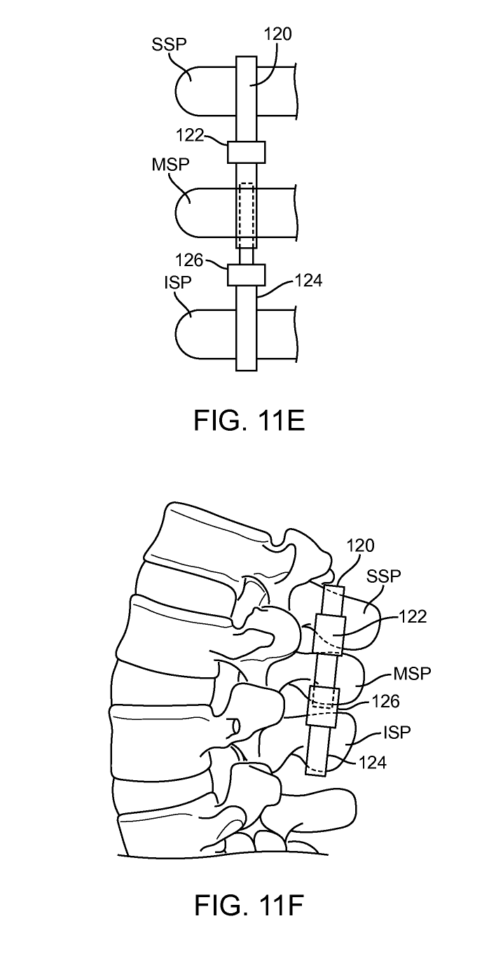

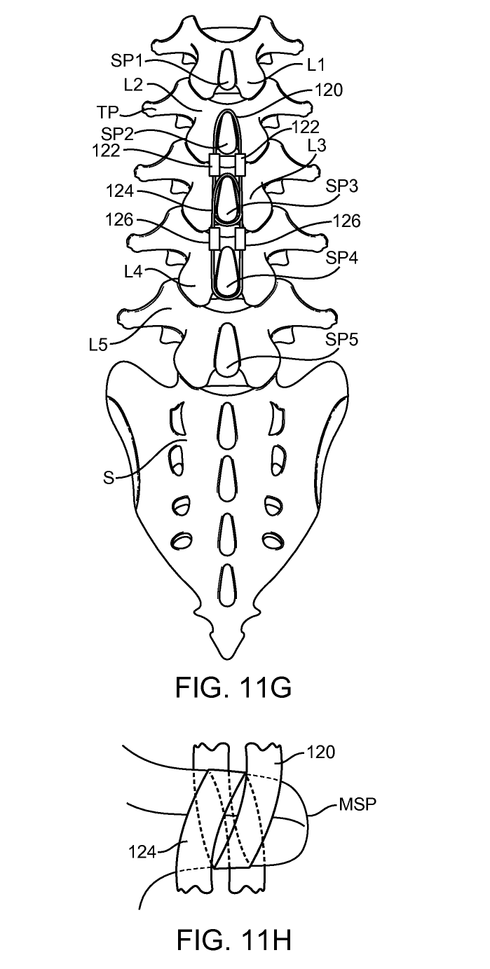

FIGS. 11A-11G illustrate various configurations of two tether structures used to restrict flexion of two spinal segments. FIG. 11A illustrates a schematic view of two tether structures 120, 124 each having a pair of compliance members 122, 126, with the tether structures placed over two adjacent pairs (SSP, MSP, and MSP, ISP) of spinous processes. The two tether structures are placed over a common MSP. FIG. 11B more clearly illustrates a sagittal view of FIG. 11A and shows the positioning of tethers around the spinous processes. A first tether structure 120 having two compliance members 122 (only one visible in this view) is disposed over a superior surface of a superior spinous process SSP and also is disposed under an inferior surface of an intermediate spinous process MSP. A second tether structure 124 having two compliance members 126 (only one visible in this view) is disposed over a superior surface of the intermediate spinous process MSP and under an inferior surface of the inferior spinous process ISP. Tether structure 120 is offset from tether structure 124 so the two tethers do not overlap with one another. FIG. 11C illustrates a posterior view of FIG. 11B, and FIG. 11D more closely illustrates how the two tether structures are disposed on the common spinous process. The compliance members within a tether structure may have the same elasticity as one another, or they may be different. Similarly, the compliance members 122 in the upper tether structure 120 may have the same elasticity as the compliance members 126 in the lower tether structure 124, or they may be different. Any number of combinations are possible. In embodiments where multiple tethers are coupled to a spinous process, such as in SP5 in FIG. 10, the first tether may be coupled to the spinous process and it is often advanced in the anterior direction in order to allow room for the second tether which will be slightly posterior to the first tether and allow both tether structures to encircle a single, intermediate spinous process. Treating two spinal segments with two discrete tether structures may allow each spinal segment to be treated with more precision than if both segments were treated with a single, contiguous structure. For example, the length and tension of each discrete tether structure may be independently adjusted, thereby more precisely providing the correct force resistant to flexion for each spinal segment. FIGS. 11A-11B show the first tether structure 120 anterior to the second tether structure 124 on the intermediate spinous process MSP. While this embodiment shows a slight gap between the two tethers, the two tethers may also be pushed against one another so there is no gap, or in some embodiments the two tethers may slightly overlap one another. In preferred embodiments, both tether structures are disposed symmetrically about the spinous processes such that side portions of each tether structure extend substantially parallel to one another on opposite sides of the spinous processes. In alternative embodiments, the first tether structure 120 may be posterior to the second tether structure 124.

FIG. 11E illustrates a schematic diagram of another tether structure configuration, and FIGS. 11F-11H illustrate sagittal and posterior views of the configuration in FIG. 11E. This embodiment is similar to that of FIGS. 11A-11B with the major difference being that the first and second tether structures now overlap with one another. It may be desirable to position the tether structures so they overlap and/or loop through one another such as shown in FIGS. 11E-11H to help secure the tether structures to the common spinous process. In this embodiment, the lower portion of the first tether structure 120 overlaps with the upper portion of tether structure 124, such that the two tether structures are looped around one another. This overlapping configuration may permit more optimal positioning of both tether structures along the spinous process, reduce the interfacial contact area of tether structures on the spinous process, and reduce the extent of iatrogenic disruption of the interspinous ligament. FIG. 11H shows the first tether structure 120 disposed partially on top of the second tether structure 124, however, one of skill in the art will appreciate that the second tether structure 124 may be positioned over the first tether structure 120.

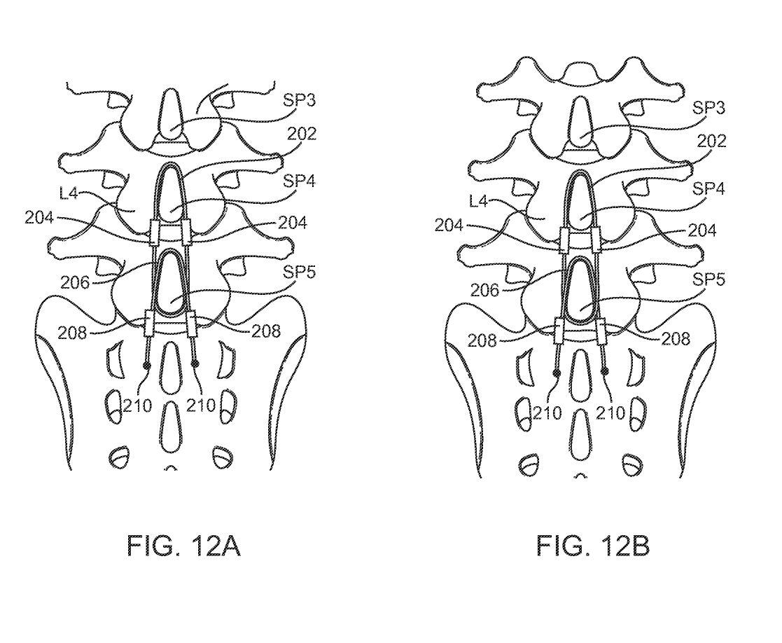

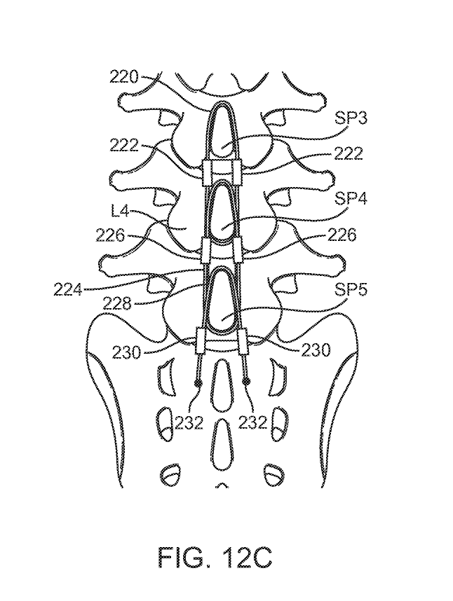

FIGS. 12A-12C illustrate still other embodiments where multiple tether structures are applied to the spinous processes or sacrum of two or more adjacent spinal segments. In FIG. 12A, a first tether structure 202 is disposed around a first superior pair of spinous processes. The first tether structure 202 has a pair of compliance members 204 and forms a contiguous loop around the first superior pair of spinous processes. A second tether structure 206 is disposed around the superior spinous process of a second inferior spinal segment. The inferior spinous process of the superior segment is the same as the superior spinous process of the inferior segment, and therefore is a common or shared spinous process. The second tether structure has a pair of compliance members 208, and two free ends 210 that are each secured to the sacrum using techniques known in the art, such as those disclosed in International Patent Application No. PCT/US2010/022767 (Attorney Docket No. 026398-001210PC), the entire contents of which are incorporated herein by reference. The tether structures and compliance members may take the form of any of those disclosed herein. Additionally, in this embodiment, the first tether structure 202 is positioned anteriorly on the spinous processes relative to the second tether structure 206, without any overlap. In alternative embodiments, the relative positions of the two tether structures may be reversed so that the first tether structure is posterior to the second tether structure.

FIG. 12B illustrates a variation of the embodiment in FIG. 12A, with the major difference being that the two tether structures are looped together around the common spinous process. Thus, in this embodiment, a lower portion of the first tether structure 202 is intertwined with an upper portion of the second tether structure 206, at the level of the common spinous process.

FIG. 12C illustrates still another exemplary embodiment where three discrete tether structures are attached to three spinal segments. A first tether structure 220 has a pair of compliance members 222, and is disposed around a superior spinal segment such that the tether structure forms a contiguous loop around a first pair of spinous processes. A second tether structure 224 also has a pair of compliance members 226, and is disposed around an intermediate spinal segment such that the tether structure forms a contiguous loop around a second pair of spinous processes. The first pair and the second pair of spinous processes share a common spinous process. A third tether structure 228 includes a pair of compliance members 230, and has an upper portion and two free ends 232 that partially encircle an inferior spinal segment. The upper portion is looped around a spinous process and the free ends 230 are attached to the sacrum using techniques such as those disclosed in International Patent Application No. PCT/US2010/022767, previously incorporated by reference. The three tether structures may be looped around one another or spaced apart as describe above, and the tether structures may take the form of any of those described herein.

In each of the embodiments disclosed herein, the tether structure limits flexion of a spinal segment. Additionally, because the tether structure is flexible and has a low elastic stiffness in compression, it does not substantially limit extension of the spinal segment. Any of the embodiments may utilize tether structures that have an elastic stiffness in compression below 3 Newtons per millimeter (N/mm). In some embodiments the elastic stiffness in compression may be below 0.5 N/mm.

It will be appreciated that numerous other combinations of continuous tether structures and discontinuous tether structures could be provided in order to effect the controlled application of elastic restraint on adjacent spinal segments in the lumbar region of the spine. Thus, the examples set forth above are not meant to be limiting on the breadth of the invention as set forth in the following claims.

What is claimed is:

1. A method for constraining spinous processes to elastically limit flexion of two or more adjacent spinal segments, said method comprising:
placing a tether structure over spinous processes of at least three adjacent vertebral bodies, or of two adjacent vertebral bodies and a sacrum, wherein the structure elastically couples an upper spinous process and a lower spinous process or sacrum so as to limit flexion therebetween without substantially limiting extension thereof, and
wherein a first portion of the tether structure extends between the upper spinous process and the lower spinous process or the sacrum, and a second portion of the tether structure extends between the upper spinous process and the lower spinous process or the sacrum, the first and the second portions disposed symmetrically on opposite sides of the spinous processes, and substantially parallel to one another.

2. A method as in claim 1, wherein the tether structure elastically couples an upper spinous process and a lower spinous process or sacrum with at least one intermediate spinous process free from coupling.

3. A method as in claim 1, wherein the tether structure elastically couples an upper spinous process, a lower spinous process or sacrum, and at least one intermediate spinous process.

4. A method as in claim 3, wherein upper spinous process, intermediate spinous process, and lower spinous process or sacrum are coupled by a single contiguous tether structure.

5. A method as in claim 3, wherein the upper spinous process, intermediate spinous process, and lower spinous process or sacrum are coupled by at least two contiguous tether structures.

6. A method as in claim 1, wherein the tether structure is disposed around a first surface of a spinous process, and the method further comprises positioning a second tether structure around a second surface of the spinous process, wherein the second surface is opposite the first surface, and wherein the second tether positioned on the spinous process such that one tether is anteriorly disposed relative to the other tether structure.

7. A method as in claim 1, wherein a lower one of the vertebral bodies is selected from the group consisting of L4, L5, and the sacrum.

8. A method as in claim 1, wherein spaces between the adjacent vertebral bodies are free from structure which would inhibit extension.

9. A method as in claim 1, wherein the tether structure comprises one or more band elements in series with one or more compliance members.

10. A method as in claim 1, wherein the tether structure comprises at least two compliance members, further comprising positioning the compliance members symmetrically to lie on opposite sides of the spinous processes.

11. A method as in claim 1, wherein the tether structure comprises at least four compliance members, further comprising positioning pairs of the compliance members symmetrically on opposite sides of the spinous processes.

12. A method as in claim 1, wherein the tether structure comprises a first compliance member having a first elasticity and a second compliance member having a second elasticity, the first compliance member superior to the second compliance member, and wherein the first elasticity is different than the second elasticity.

13. A method as in claim 1, wherein the tether structure comprises a first pair of compliance members and a second pair of compliance members, wherein the first pair of compliance members each have a first elasticity and the second pair of compliance members each have a second elasticity, the first elasticity being different than the second elasticity, and wherein the first pair of compliance members are superior to the second pair of compliance members.

14. A method as in claim 1, wherein the tether structure is disposed over the upper spinous process, the lower spinous process and an intermediate spinous process disposed therebetween, and wherein the tether structure comprises a first loop encircling the lower spinous process and the intermediate spinous process so as to substantially prevent flexion therebetween, and wherein the tether structure comprises a second loop superior to the first loop, the second loop having one or more compliance members and disposed over the upper spinous process and coupled with the first loop so as to provide a force resistant to flexion of a superior spinal segment relative to the inferior spinal segment.

15. A method as in claim 1, wherein the tether structure provides an elastic stiffness in compression below 3 N/mm.

16. A method as in claim 15, wherein the elastic stiffness in compression is below 0.5 N/mm.

17. A spinal implant comprising a contiguous tether structure adapted to circumscribe at least two non-adjacent spinous processes or an anchor location on a sacrum and one non-adjacent spinous process, wherein at least a portion of the tether structure provides an elastic resistance to elongation in response to an elongation force which results from flexion of the spinal segments between the non-adjacent spinous processes or the one non-adjacent spinous process and the sacrum, and wherein the tether structure limits flexion therebetween without substantially limiting extension therebetween, and
wherein a first portion of the tether structure extends between the non-adjacent spinous processes or between the one non-adjacent spinous process and the sacrum, and a second portion of the tether structure extends between the non-adjacent spinous processes or between the one non-adjacent spinous process and the sacrum, the first and the second portions disposed symmetrically on opposite sides of the spinous processes, and substantially parallel to one another.

18. A spinal implant as in claim 17, further comprising at least two compliance members positioned symmetrically to lie on opposite sides of the spinous processes.

19. A spinal implant as in claim 17, further comprising at least four compliance members positioned symmetrically to lie on opposite sides of the spinous process.

20. A spinal implant as in claim 17, further comprising a first compliance member having a first elasticity and a second compliance member having a second elasticity, the first compliance member superior to the second compliance member, and wherein the first elasticity is different than the second elasticity.

21. A spinal implant as in claim 17, further comprising a first pair of compliance members and a second pair of compliance members, wherein the first pair of compliance members each have a first elasticity and the second pair of compliance members each have a second elasticity, the first elasticity being different than the second elasticity, and wherein the first pair of compliance members are superior to the second pair of compliance members.

22. A spinal implant as in claim 17, wherein the contiguous tether structure is continuous to loop over said non-adjacent spinous processes.

23. A spinal implant as in claim 17, wherein the contiguous tether structure is discontinuous and has two ends, each end having an anchor for attachment to bone.

24. A spinal implant as in claim 17, wherein the tether structure is disposed over an upper spinous process, a lower spinous process and an intermediate spinous process disposed therebetween, and wherein the tether structure comprises a first loop encircling the lower spinous process and the intermediate spinous process so as to substantially prevent flexion therebetween, and wherein the tether structure comprises a second loop superior to the first loop, the second loop having one or more compliance members and disposed over the upper spinous process and coupled with the first loop so as to provide a force resistant to flexion of a superior spinal segment relative to the inferior spinal segment.

25. A spinal implant as in claim 17, wherein the tether structure provides an elastic stiffness in compression below 3 N/mm.

26. A spinal implant as in claim 25, wherein the elastic stiffness in compression is below 0.5 N/mm.

27. A system comprising a spinal implant as in claim 17, and at least one additional contiguous tether structure adapted to circumscribe two adjacent or non-adjacent spinous processes or a sacrum.

28. A system as in claim 27, wherein the additional contiguous tether structure is continuous to loop over said non-adjacent spinous processes.

29. A system as in claim 27, wherein the additional contiguous tether structure is discontinuous and has two ends, each end having an anchor for attachment to bone.

30. A system as in claim 27, wherein the contiguous tether structure is disposed around a first surface of a spinous process, and the additional tether structure is disposed around a second surface of the spinous process, wherein the second surface is opposite the first surface, and wherein the additional tether structure is positioned on the spinous process such that one tether is anteriorly disposed relative to the other tether structure.

31. A method for constraining spinous processes to elastically limit flexion of two or more adjacent spinal segments, said method comprising:
placing a first tether structure over a superior spinous process and an inferior spinous process of a first spinal segment, wherein the first tether structure elastically couples the superior spinous process and the inferior spinous process so as to limit flexion therebetween without substantially limiting extension thereof,
wherein a first portion of the first tether structure extends between the superior spinous process and the inferior spinous process of the first spinal segment, and a second portion of the first tether structure extends between the superior spinous process and the inferior spinous process of the first spinal segment, the first and the second portions disposed symmetrically on opposite sides of the spinous processes, and substantially parallel to one another; and
placing a second tether structure over a superior spinous process and an inferior spinous process or a sacrum of a second spinal segment, wherein the second tether structure elastically couples the superior spinous process and the inferior spinous process or the sacrum of the second spinal segment so as to limit flexion therebetween without substantially limiting extension thereof,
wherein a first portion of the second tether structure extends between the superior spinous process and the inferior spinous process or the sacrum of the second spinal segment, and a second portion of the second tether structure extends between the superior spinous process and the inferior spinous process or the sacrum of the second spinal segment, the first and the second portions disposed symmetrically on opposite sides of the spinous processes, and substantially parallel to one another, and
wherein the first spinal segment is adjacent and superior to the second spinal segment.

32. The method of claim 31, wherein one of the first or the second tether structures is positioned anteriorly relative to the other tether structure.

33. The method of claim 31, wherein the first spinal segment and the second spinal segment share a common spinous process.

34. The method of claim 31, wherein the inferior spinous process of the first spinal segment and the superior spinous process of the second spinal segment are the same spinous process.

35. The method of claim 34, wherein placing the first tether structure and placing the second tether structure comprise placing the tether structures without engaging or overlapping one another.

36. The method of claim 34, wherein placing the first tether structure and placing the second tether structure comprise looping the tether structures through each other whereby the tether structures encircle the common spinous process.

37. The method of claim 31, wherein the first tether structure and the second tether structures each comprise a discrete contiguous loop.

38. The method of claim 31, wherein at least one of the tether structures comprise two compliance members, the method further comprising positioning the compliance members symmetrically to lie on opposite sides of the spinous processes.

39. A system for restricting flexion of two or more adjacent spinal segments, said system comprising:
a first contiguous tether structure comprising an upper tether portion and a lower tether portion, wherein the first tether structure is configured to surround a first spinal segment, the first spinal segment having a superior spinous process and an inferior spinous process, wherein the upper tether portion is positioned around the superior spinous process, and the lower tether portion is positioned around the inferior spinous process, and
wherein the first tether structure provides an elastic resistance to flexion of the first spinal segment without substantially limiting extension thereof; and
a second contiguous tether structure comprising an upper tether portion and a lower tether portion, wherein the second tether structure is configured to surround a second spinal segment, the second spinal segment having a superior spinous process and an inferior spinous process or a sacrum, wherein the upper tether portion of the second tether structure is positioned around the superior spinous process of the second spinal segment, and the lower tether portion of the second tether structure is positioned around the inferior spinous process of the second spinal segment or coupled to the sacrum, and
wherein the second tether structure provides an elastic resistance to flexion of the second spinal segment without substantially limiting extension thereof.

40. The system of claim 39, wherein the first tether structure comprises a first side portion, and a second side portion, wherein the first side portion and the second side portions extend from the upper spinous process and the lower spinous process of the first spinal segment, and wherein the first and the second side portions are disposed symmetrically on opposite sides of the spinous processes, and substantially parallel to one another.

41. The system of claim 39, wherein the second tether structure comprises a first side portion, and a second side portion, wherein the first side portion and the second side portion extend from the upper spinous process and the lower spinous process of the second spinal segment, and wherein the first and the second side portions are disposed symmetrically on opposite sides of the spinous processes, and substantially parallel to one another.

42. The system of claim 39, wherein the first tether structure is discrete and unconnected to the second tether structure.

43. The system of claim 39, wherein the first tether structure is disposed anterior to the second tether structure.

44. The system of claim 39, wherein the first tether structure is disposed posterior to the second tether structure.

45. The system of claim 39, wherein a portion of the first tether structure overlaps with a portion of the second tether structure.

46. The system of claim 45, wherein the first tether structure overlaps with the second tether structure such that the tether structures loop through each other, each of the tether structures engaged with a common spinous process.

47. The system of claim 46, wherein each of the first and the second tether structures overlap with one another and engage the common spinous process at substantially the same distance posterior from an origin of the common spinous process relative to the lamina.

48. The system of claim 39, wherein the first tether structure comprises a first and a second compliance member, the compliance members disposed symmetrically on opposite sides of the spinous processes, and substantially parallel to one another.

49. The system of claim 48, wherein the first compliance member comprises a first elasticity, and the second compliance member comprises a second elasticity, and wherein the first elasticity is substantially the same as the second elasticity.

50. The system of claim 39, wherein the second tether structure comprises a first and a second compliance member, the compliance members disposed symmetrically on opposite sides of the spinous processes, and substantially parallel to one another.

51. The system of claim 50, wherein the first compliance member comprises a first elasticity, and the second compliance member comprises a second elasticity, and wherein the first elasticity is substantially the same as the second elasticity.

52. The system of claim 39, wherein the first tether structure comprises a first and a second compliance member, the first and the second compliance members disposed symmetrically on opposite sides of the spinous processes, and substantially parallel to one another, and
wherein the second tether structure comprises a first and a second compliance member, the first and the second compliance members of the second tether structure being disposed symmetrically on opposite sides of the spinous processes, and substantially parallel to one another, and
wherein the elasticity of the first and the second compliance members of the first tether structure is different than the elasticity of the first and the second compliance members of the second tether structure.

53. The system of claim 39, wherein either the first tether structure or the second tether structure provide an elastic stiffness in compression below 3 N/mm.

54. The system of claim 53, wherein the elastic stiffness in compression is below 0.5 N/mm.

55. The system of claim 39, wherein the first spinal segment and the second spinal segment share a common spinous process.

56. The system of claim 39, wherein the inferior spinous process of the first spinal segment and the superior spinous process of the second spinal segment are the same spinous process.
